(19) United States
(12) Patent Application Publication
(10) Pub. No.: US 2023/0122001 A1
(43) Pub. Date: Apr. 20, 2023

(54) POLYMER

(71) Applicant: Sumitomo Chemical Co., Ltd., Tokyo (JP)

(72) Inventor: Kiran Kamtekar, Godmanchester (GB)

(73) Assignee: Sumitomo Chemical Co., Ltd., Tokyo (JP)

(21) Appl. No.: 17/913,680

(22) PCT Filed: Mar. 23, 2021
(86) PCT No.: PCT/EP2021/057481

§ 371 Date: Sep. 22, 2022

(30) Foreign Application Priority Data
Mar. 24, 2020 (GB) 2004274.3

(51) Int. Cl. H10K 85/10; H10K 30/30; C08G 61/12

(52) U.S. Cl. CPC H01L 51/0036; H01L 51/4253; C08G 61/122; C08G 61/126

(57) ABSTRACT

A polymer comprising an electron-donating repeat unit of formula (I) and an electron-accepting repeat unit: -(A)ₙ- (I) wherein A in each occurrence is independently a group of formula (II): Y in each occurrence is independently O or S. Z is O, S or NR³ wherein R³ is H or a substituent. R¹ in each occurrence is independently H or a substituent. R² in each occurrence is independently a substituent, n is at least 2. The polymer may be used as an electron-donating polymer in combination with an electron-accepting material in a bulk heterojunction layer of an organic photodetector.

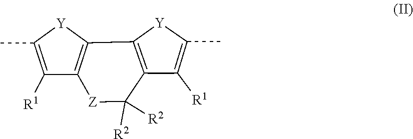

### BACKGROUND

Donor-acceptor (D-A) polymers are known for use in organic photovoltaic devices.

Chu et al, “Dithieno[3,2-b:2′,3′-d]pyran-containing organic D-n-A sensitizers for dye-sensitized solar cells”, discloses D-π-A sensitisers incorporating a dithieno[3,2-b:2′,3′-d]pyran and dye-sensitised solar cells containing these sensitisers.

CN104478900 discloses a monomer for preparing a donor material used in a polymer solar cell. The monomer is a compound containing two lactam six-membered rings and the lactam structure is connected by a single bond or a conjugated bridge.

EP2767553 discloses a polymer comprising a constituent unit represented by Formula (1) and a constituent unit represented by Formula (2):

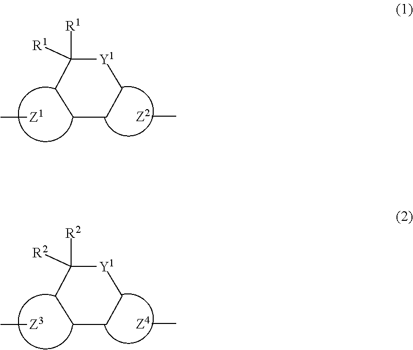

### SUMMARY

According to some embodiments, the present disclosure provides a polymer comprising an electron-donating repeat unit of formula (I) and an electron-accepting repeat unit:

-(A)ₙ- (I)

wherein A in each occurrence is independently a group of formula (II):

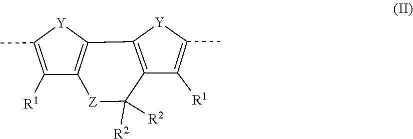

wherein:

Y in each occurrence is independently O or S;

Z is O, S or NR³ wherein R³ is H or a substituent;

R¹ in each occurrence is independently H or a substituent;

R² in each occurrence is independently H or a substituent; and

n is at least 2.

Optionally, n is 2.

Optionally, each R¹ is H.

Optionally, each R² is independently selected from the group consisting of:
linear, branched or cyclic C₁₋₂₀ alkyl wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1.

Optionally, each Y is S.

Optionally, the electron-accepting repeat unit is selected from formulae (III)-(XIII):

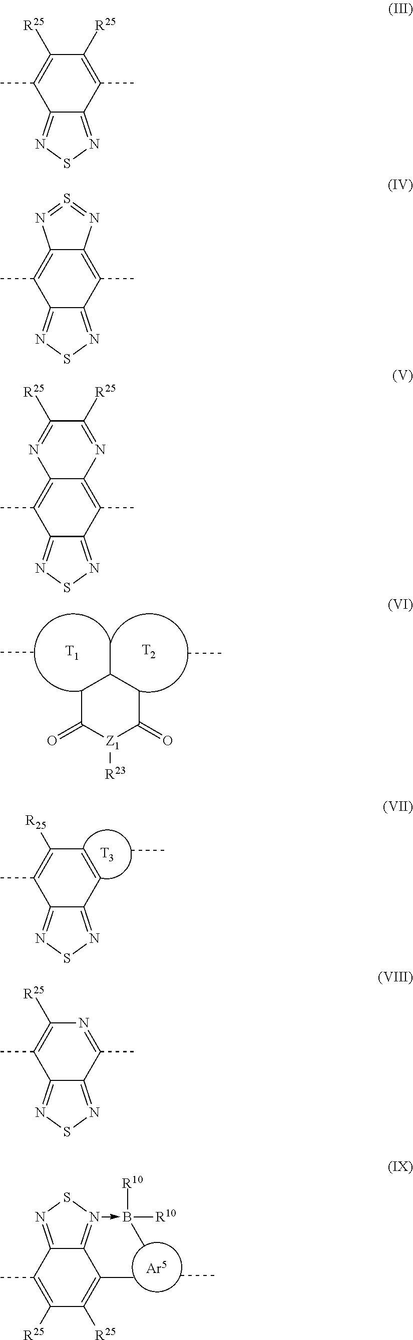

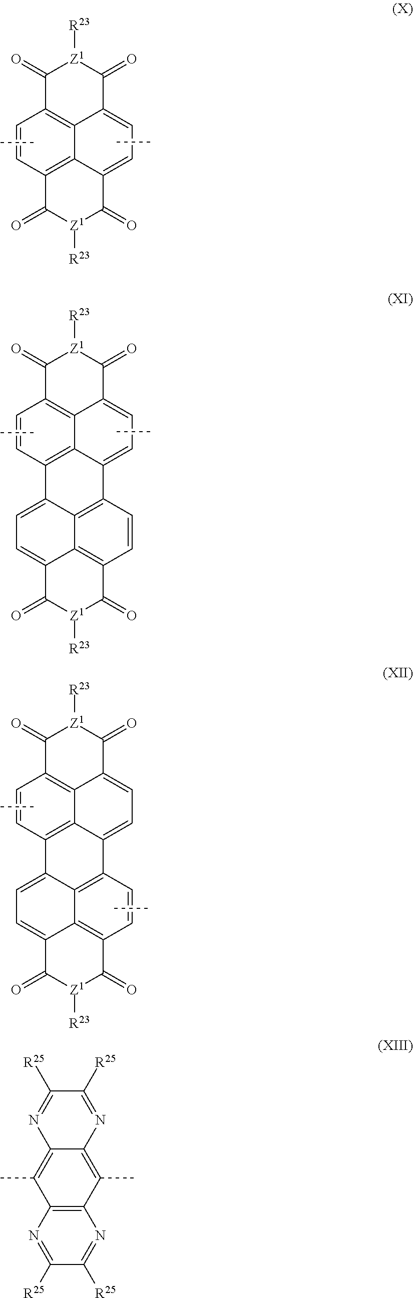

wherein R²³ in each occurrence is H or a substituent; R²⁵ in each occurrence is H or a substituent wherein two R²⁵ groups bound to adjacent carbon atoms may be linked to form a substituted or unsubstituted ring; Z¹ is N or P; T¹, T² and T³ each independently represent an aryl or a heteroaryl ring which may be fused to one or more further rings; R¹⁰ in each occurrence is a substituent; and Ar⁵ is an arylene or heteroarylene group which is unsubstituted or substituted with one or more substituents.

According to some embodiments, the present disclosure provides a composition comprising a polymer as described herein and an electron-accepting material.

According to some embodiments, the present disclosure provides an organic electronic device comprising an active layer comprising compound or composition as described herein.

Optionally, the organic electronic device is an organic photoresponsive device comprising a bulk heterojunction layer comprising the composition described herein disposed between an anode and a cathode.

Optionally, the organic photoresponsive device is an organic photodetector.

According to some embodiments, the present disclosure provides a photosensor comprising a light source and an organic photodetector as described herein wherein the photosensor is configured to detect light emitted from a light source.

Optionally, the light source emits light having a peak wavelength of at least 850 nm.

According to some embodiments, the present disclosure provides a formulation comprising a polymer or a composition as described herein dissolved or dispersed in one or more solvents.

According to some embodiments, the present disclosure provides a method of forming an organic electronic device as described herein wherein formation of the active layer comprises deposition of a formulation as described herein onto a surface and evaporation of the one or more solvents.

According to some embodiments, the present disclosure provides a compound of formula (Im):

X-(A)ₙ-X (Im)

wherein:

X in each occurrence is independently selected from the group consisting of halogen, —OSO₂R⁴ wherein R⁴ is an optionally substituted C₁₋₁₂ alkyl group or optionally substituted aryl group; boronic acid and esters thereof; and —SnR⁵₃ wherein R⁵ independently in each occurrence is a C₁₋₁₂ hydrocarbyl group; and

A and n are as described with respect to Formula (I).

According to some embodiments, the present disclosure provides a method of forming a polymer as described herein comprising polymerisation of the compound of formula (Im) and a compound for forming the electron-accepting repeat unit.

### DESCRIPTION OF DRAWINGS

The disclosed technology and accompanying figures describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that the peak absorption wavelength of a donor-acceptor polymer may be increased by providing two or more adjacent donor units between acceptor units of the polymer.

The polymer has a repeat unit of formula (I):

-(A)ₙ- (I)

A in each occurrence is independently a group of formula (II):

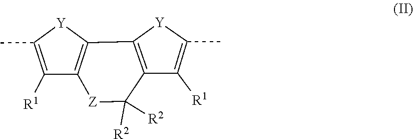

Y in each occurrence is independently O or S, preferably S.

Z is O, S or NR³ wherein R³ is H or a substituent.

R¹ in each occurrence is independently H or a substituent.

R² in each occurrence is independently H or a substituent, preferably a substituent.

n is at least 2, optionally 2, 3, 4 or 5. Preferably, n is 2.

Each group A of formula (I) may be the same or different. In some embodiments, Z of at least one of the n groups is O or S and at least one other of the n groups is NR³. The A groups may be linked in the same orientation or different orientations.

Preferably, each R² is independently selected from the group consisting of:
linear, branched or cyclic C₁₋₂₀ alkyl wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1.

Optionally, each R¹ is independently selected from H and a substituent as described with reference to R². Preferably, each R¹ is H.

Exemplary repeat units of formula (I) include, without limitation, repeat units of formulae (I-A) to (I-L):

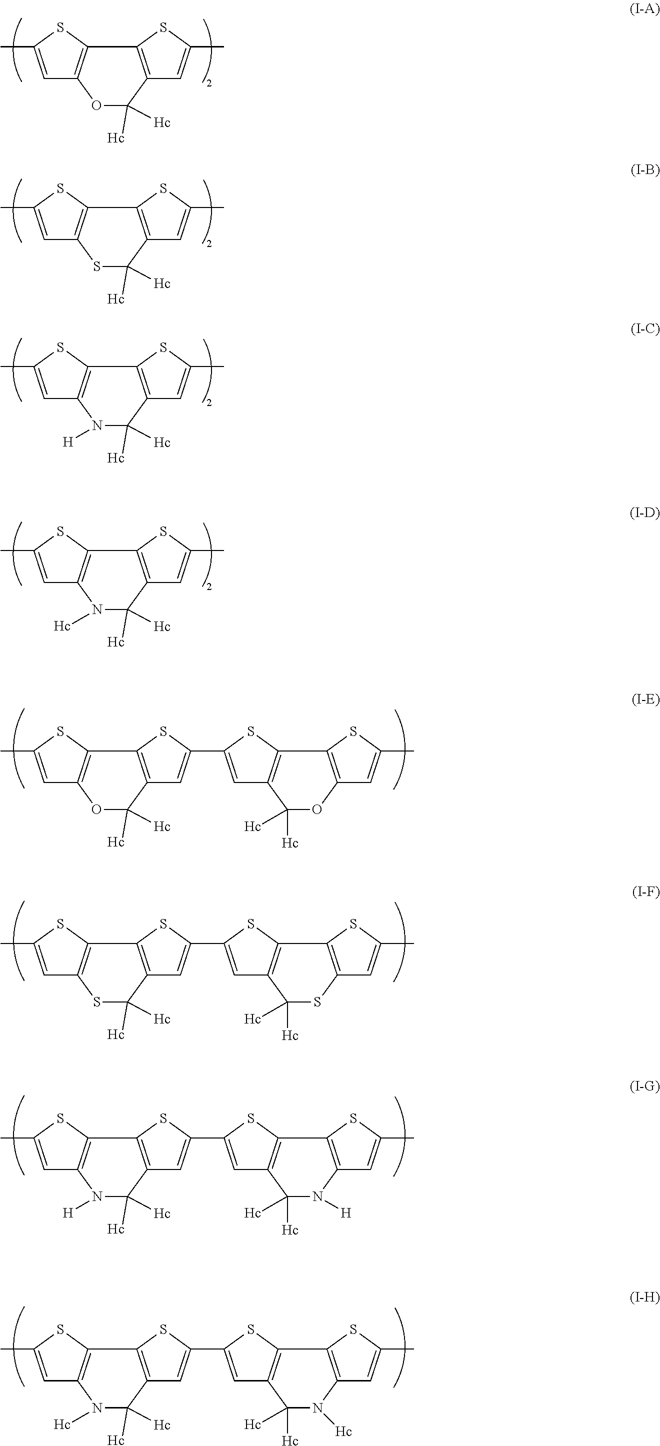

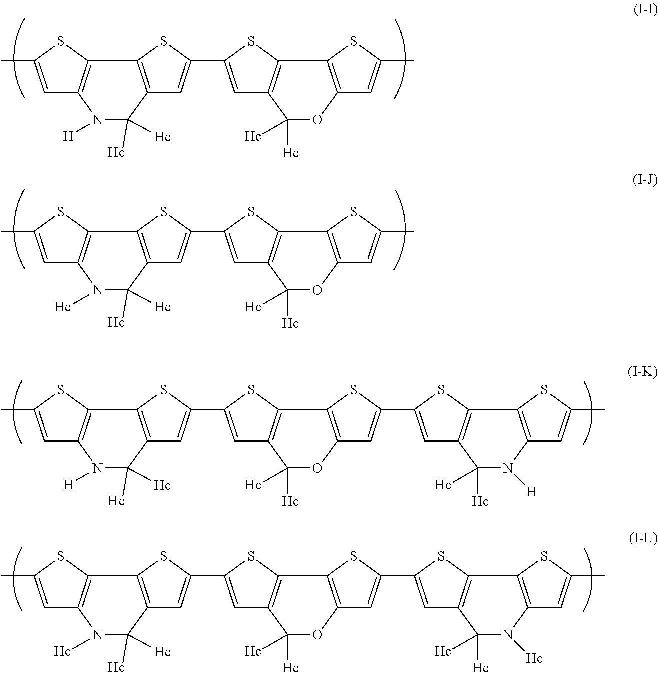

wherein He in each occurrence is independently a C₁₋₂₀ hydrocarbyl group, e.g. C₁₋₂₀ alkyl, unsubstituted aryl, or aryl substituted with one or more C₁₋₁₂ alkyl groups. The aryl group is preferably phenyl.

Groups A of Formulae (I-A)-(I-D) are the same and are linked in the same orientation.

Groups A of Formulae (I-E)-(I-H) are the same and are linked in different orientations.

Groups A of Formulae (I-I)-(I-L) are different and include A groups with different orientations.

The polymer contains electron-donating repeat units of formula (I) and an electron-accepting repeat unit. The electron-accepting repeat unit has a LUMO level that is deeper (i.e. further from vacuum) than the electron-donating repeat unit, preferably at least 1 eV deeper. The LUMO levels of repeat units of formula (I) and electron-accepting repeat units may be as determined by modelling the LUMO level of each repeat unit, in which bonds to adjacent repeat units are replaced with bonds to a hydrogen atom. Modelling may be performed using Gaussian09 software available from Gaussian using Gaussian09 with B3LYP (functional) and LACVP* (Basis set).

Optionally, the electron-accepting repeat unit is selected from formulae (III)-(XIII):

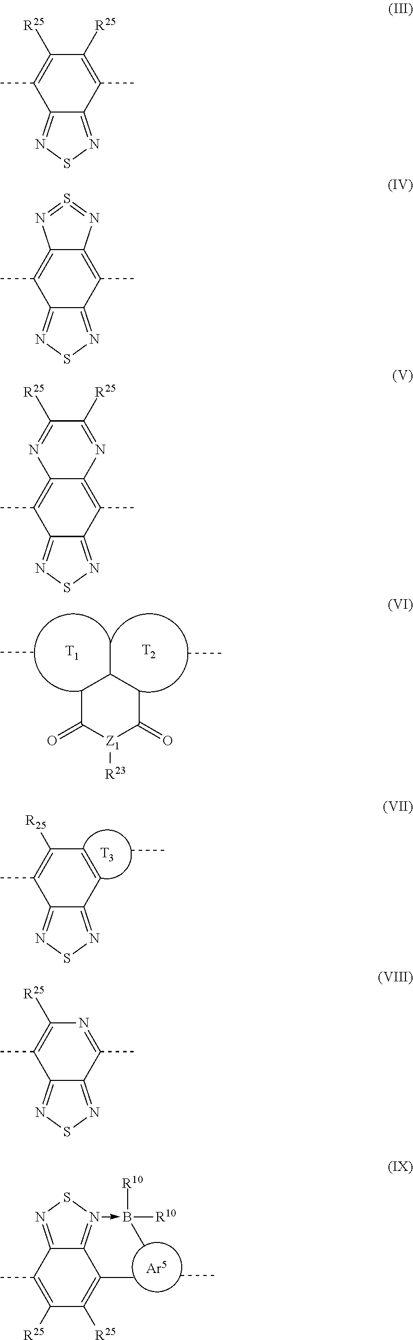

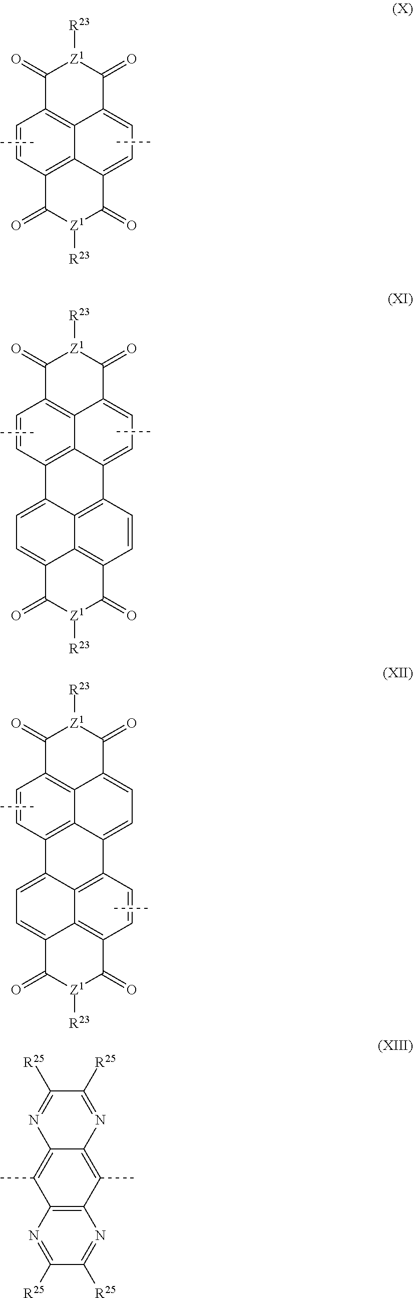

R²³ in each occurrence is H or a substituent, optionally H or C₁₋₁₂ alkyl wherein one or more non-adjacent, non-terminal C atoms may be replaced with O, S, COO or CO and one or more H atoms of the alkyl may be replaced with F.

By “non-terminal” C atom of an alkyl group as used herein is meant a C atom of the alkyl other than the methyl C atom of a linear (n-alkyl) chain or the methyl C atoms of a branched alkyl chain.

R²⁵ in each occurrence is independently H; F; C₁₋₁₂ alkyl wherein one or more non-adjacent, non-terminal C atoms may be replaced with O, S, COO or CO and one or more H atoms of the alkyl may be replaced with F; or an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO. In the case where two R²⁵ groups are bound to adjacent carbon atoms, the two R²⁵ groups may be linked to form a substituted or unsubstituted ring, preferably a substituted or unsubstituted aryl or heteroaryl ring. Where present, substituents of such a ring are optionally selected from F, CN, NO₂ and C₁₋₁₂ alkyl wherein one or more non-adjacent, non-terminal C atoms may be replaced with O, S, COO or CO and one or more H atoms of the alkyl may be replaced with F

Z¹ is Nor P.

T¹, T² and T³ each independently represent an aryl or a heteroaryl ring, optionally benzene, which may be fused to one or more further rings. Substituents of T¹, T² and T³, where present, are optionally selected from non-H groups of R²⁵. Optionally, T³ is benzothiadiazole and the repeat unit of formula (VII) has formula (VIIa):

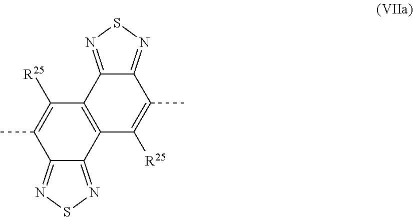

R¹⁰ in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Optionally, the polymer has an absorption spectrum having a peak at a wavelength greater than about 850 nm. The absorption spectrum may be as measured in solution, optionally toluene solution, using a Cary 5000 UV-vis-IR spectrometer. Measurements may be taken from 175 nm to 3300 nm using a PbSmart NIR detector for extended photometric range with variable slit widths (down to 0.01 nm) for optimum control over data resolution.

Absorption intensity is plotted vs. incident wavelength to generate an absorption spectrum. A method for measuring film absorption, may comprise measuring a 15 mg/ml solution in a quartz cuvette and comparing to a cuvette containing the solvent only.

Preferably, the polystyrene-equivalent number-average molecular weight (Mn) measured by gel permeation chromatography of a polymer as described herein is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

Polymer Synthesis and Monomers

A polymer as described herein may be formed by polymerising a monomer for forming a repeat unit of formula (I) and a monomer for forming an electron-accepting repeat unit. The polymerisation method includes, without limitation, methods for forming a carbon-carbon bond between an aromatic carbon atom of a donor unit of formula (I) and an aromatic carbon atom of an acceptor unit.

The monomer for forming a repeat unit of formula (I) may be a compound of formula (Im):

X-(A)ₙ-X (Im)

wherein A and n are as described with reference to Formula (I) and X in each occurrence is independently a leaving group.

Optionally, each X is selected from the group consisting of halogen, —OSO₂R⁴ wherein R⁴ is an optionally substituted C₁₋₁₂ alkyl group or optionally substituted aryl group; boronic acid and esters thereof; and —SnR⁵₃ wherein R⁵ independently in each occurrence is a C₁₋₁₂ hydrocarbyl group.

Suitable polymerisation methods include, without limitation, Suzuki polymerisation and Stille polymerisation. Suzuki polymerisation is described in, for example, WO 00/53656.

The monomers and polymerisation method may be selected such that the monomer for forming the donor repeat unit of formula (I) reacts only with the monomer for forming the acceptor repeat unit, thereby forming a D-A copolymer.

In some embodiments, each X may be one of: (i) a halogen or —OSO₂R⁴; or (ii), a boronic acid or ester, and the monomer for forming the electron-accepting repeat unit may be substituted with the other of (i) and (ii).

In some embodiments, each X may be one of: (i) a halogen or —OSO₂R⁴; and (iii) —SnR⁵₃, and the monomer for forming the electron-accepting repeat unit may be substituted with the other of (i) and (iii).

Optionally, R⁴ in each occurrence is independently a C₁₋₁₂ alkyl group which is unsubstituted or substituted with one or more F atoms; or phenyl which is unsubstituted or substituted with one or more F atoms.

Optionally, R⁵ is selected from the group consisting of C₁₋₁₂ alkyl; unsubstituted phenyl; and phenyl substituted with one or more C₁₋₆ alkyl groups.

A halogen leaving group is preferably Br or I.

—OSO₂R⁴ is preferably tosylate or triflate.

Exemplary boronic esters have formula (XIV):

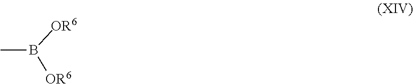

wherein R⁶ in each occurrence is independently a C₁₋₂₀ alkyl group wherein one or more non-adjacent C atoms may be replaced with C═O, O, S or NR⁷ wherein R⁷ in each occurrence is a C₁₋₁₂ hydrocarbyl group, * represents the point of attachment of the boronic ester to an aromatic ring of the monomer, and the two groups R⁶ may be linked to form a ring which is unsubstituted or substituted with one or more substituents, e.g. one or more C₁₋₆ alkyl groups or hydroxy-C₁₋₆ alkyl groups. In a preferred embodiment, the two groups R⁶ are linked, e.g. to form:

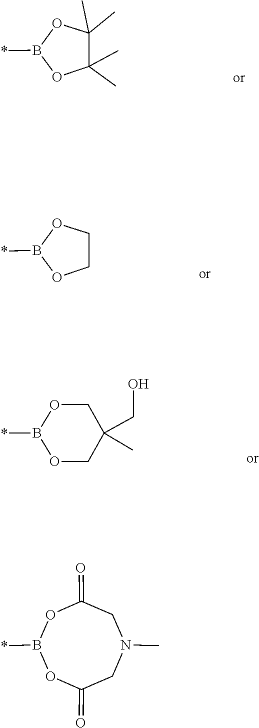

Compositions

The polymer may be part of a composition comprising or consisting of an electron-accepting (n-type) material and an electron-donating (p-type) material wherein the polymer is the electron-donating material. The composition may comprise one or more further materials, e.g. one or more further electron-donating materials and/or one or more further electron-accepting materials.

The electron-accepting material has a LUMO level that is deeper (i.e. further from vacuum) than the LUMO of the electron-donating polymer. Optionally, the gap between the HOMO level of the electron-donating polymer and the LUMO level of the electron-accepting material is less than 1.4 eV. Unless stated otherwise, HOMO and LUMO levels of materials as described herein are as measured by square wave voltammetry (SWV). Preferably, the electron-accepting material and the electron-donating polymer form a type II interface.

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels of a polymer as described herein by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

The apparatus to measure HOMO or LUMO energy levels of a material in solution by SWV may comprise a cell containing tertiary butyl ammonium perchlorate or tertiary butyl ammonium hexafluorophosphate in an acetonitrile:toluene mix (1:1); a glassy carbon working electrode; a platinum counter electrode and a leak free Ag/AgCl reference electrode.

For measurement of a polymer film, ferrocene is added directly to the existing cell at the end of the experiment for calculation purposes where the potentials are determined for the oxidation and reduction of ferrocene versus Ag/AgCl using cyclic voltammetry (CV). The same is done for a solution except that ferrocene is added to a fresh cell of identical solvent composition.

For a solution, the sample is dissolved in Toluene (3 mg/ml) and added directly to the cell.

The sample is dissolved in Toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data in the case of a polymer film, or from an average of 3 consecutive measurements of both HOMO and LUMO sweeps in the case of a solution.

All experiments are run under an Argon gas purge.

In some embodiments, the weight ratio of the electron donor material(s) comprising or consisting of a polymer as described herein to the acceptor material(s) is from about 1:0.5 to about 1:2. In some preferred embodiments, the weight ratio of the donor materials to the acceptor material(s) is about 1:1.1 to about 1:2. In some preferred embodiments, the weight of the donor materials is greater than the weight of the acceptor material(s).

The, or each, electron acceptor material is preferably a non-polymeric compound. Preferably, the non-polymeric compound has a molecular weight of less than 5,000 Daltons, optionally less than 3,000 Daltons.

The electron acceptor material may be a fullerene or a non-fullerene

Non-fullerene acceptors are described in, for example, Cheng et al, “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F.

Exemplary fullerene electron acceptor materials are C₆₀, C₇₀, C₇₆, C₇₈ and C₈₄ fullerenes or a derivative thereof including, without limitation, PCBM-type fullerene derivatives (including phenyl-C61-butyric acid methyl ester (C₆₀PCBM), TCBM-type fullerene derivatives (e.g. tolyl-C61-butyric acid methyl ester (C₆₀TCBM)), and ThCBM-type fullerene derivatives (e.g. thienyl-C61-butyric acid methyl ester (C₆₀ThCBM).

Organic Electronic Devices

A polymer or composition as described herein may be provided as an active layer of an organic electronic device. In a preferred embodiment, a bulk heterojunction layer of an organic photoresponsive device, more preferably an organic photodetector, comprises a composition as described herein.

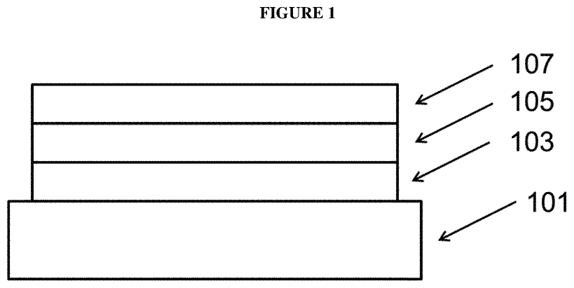

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent.

Each transparent electrode preferably has a transmittance of at least 70%, optionally at least 80%, to wavelengths in the range of 750-1000 nm. The transmittance may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer.

In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm 2, less than about 0.75 cm², less than about 0.5 cm² or less than about 0.25 cm². The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron acceptor material. The bulk heterojunction layer may consist of these materials or may comprise one or more further materials, for example one or more further electron donor materials and/or one or more further electron acceptor materials.

Formulations

A layer containing a polymer or composition as described herein may be formed by depositing a formulation containing a polymer or a composition as described herein dissolved or dispersed in one or more solvents and evaporating the one or more solvents.

The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene.

The formulation may comprise further components. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

Applications

A circuit may comprise an organic photodetector as described herein connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 850 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject.

The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Monomer Example 1

Monomer Example 1 may be formed according to Scheme 1:

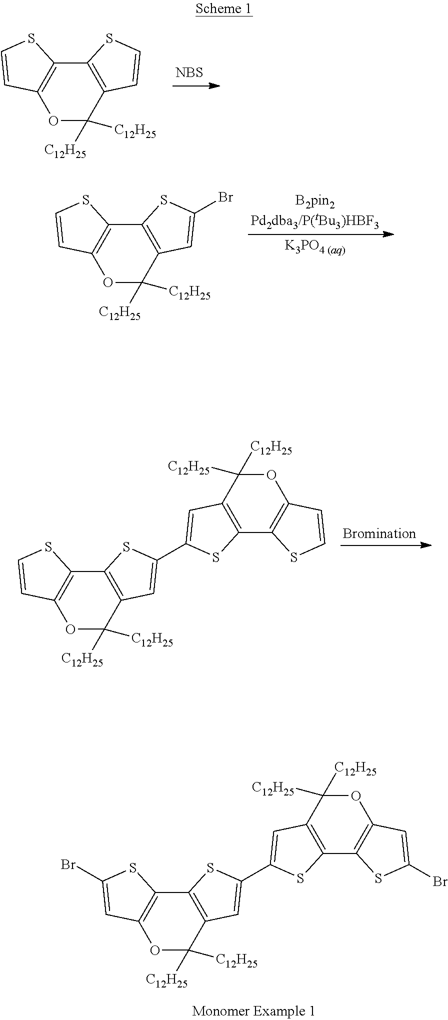

### Monomer Example 1 Stage 1

A nitrogen-purged solution of 5,5-bisdodecyldithieno[3,2-b:2′,3′-d]pyran (1 g, 1.88 mmol) in THF (19 mL) was cooled down to −35° C. (MeCN/dry ice). To this solution NBS (1.71 mmol, 0.31 g) was added in 3 portions. The reaction was allowed to stir at the same temperature for 1 h. The reaction was warmed up to room temperature and extracted with water and heptane.

Combined organic layers were dried with MgSO₄, filtered and the solvent was removed in vacuum. The crude product was a yellow oil (0.95 g) which was used in the next step without further purification.

### Monomer Example 1 Stage 2

A solution of stage 1 material (10.1 g, 16.56 mmol) in THF (82 mL) and aqueous solution of K₃PO₄ (3 M, 82 mL) was degassed for 0.5 h. To this mixture, Pd₂(dba)₃ (0.61 g, 0.66 mmol), [t-Bu₃PH]BF₄ (0.77 g, 2.65 mmol,) and bis(pinacolato)diboron (2.1 g, 8.28 mmol) were added. The reaction mixture was further degassed for 5 mins and then reaction heated to 80° C. for 2 h. T.L.C. analysis show the reaction was complete and the reaction was allowed to cool to room temperature and then the product was extracted with ethyl acetate and water. Combined organic layers were dried with MgSO₄, filtered and concentrated in vacuum. The crude product was purified by silica gel column chromatography using heptane as an eluent. The product containing fractions were combined and concentrated in vacuum to give the stage 2 material as a yellow powder (5.49 g) with ˜99% purity as determined by HPLC.

### Monomer Example 1 Stage 3

Stage 2 material (2.46 g, 2.32 mmol) was dissolved in THF (39 mL) under nitrogen. The solution was cooled down to 0° C. and NBS (0.78 g, 4.41 mmol) was added to the solution in 5 portions. The reaction was stirred at the same temperature for 1 h. The reaction was then quenched with water and extracted with water and DCM. The organic layers were combined, dried with MgSO₄, filtered and concentrated in vacuum. The rude product was purified by silica gel column chromatography using heptane as an eluent. Monomer example 1 was obtained as an orange solid (1.5 g) with 98.7% purity.

### Monomer Example 2

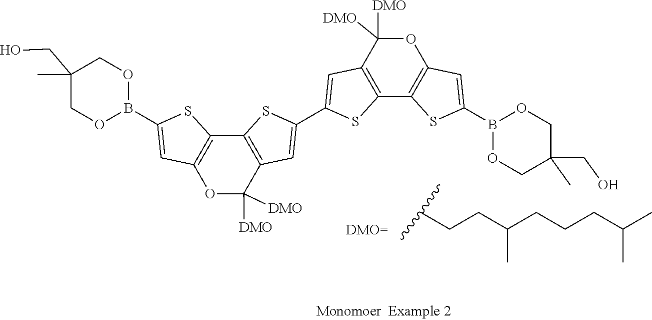

### Monomer Example 2 Stage 1

Synthesis carried out as for Monomer example 1 stage 1 yielding 6.11 g of crude material (100% yield) that was used without further purification

### Monomer Example 2 Stage 2

Synthesis was carried out as for Monomer example 1 stage 2 yielding 4.57 g (87% yield) of a dark orange oil with 99.2% HPLC purity

### Monomer Example 2 Stage 3

Stage 2 material (2 g, 2.1 mmol) and TMEDA (0.32 mL, 2.1 mmol) were dissolved in dry THF (16 mL) and cooled down to −78° C. (acetone/CO₂). N-Butyllithium (2.1 mL, 2.5 M, 5.2 mmol) was added dropwise and the reaction mixture stirred for 2 h. Triisopropylborate (1.4 mL, 5.9 mmol) was added dropwise and the reaction was stirred at −78° C. for a further 1 h before being allowed to warm to room temperature. A nitrogen-purged portion of acetic acid (11%, 23 mL) was added and the mixture was stirred for 10 mins. Nitrogen-purged toluene (40 mL) was added and the aqueous layer was removed before 1,1,1-tris(hydroxymethyl)ethane (0.76 g, 6.3 mmol) and magnesium sulfate were added and the mixture was stirred overnight. The mixture was filtered through celite and the solvent removed. The resulting crude material was recrystallized from toluene/heptane to give and orange solid (0.4 g) with 98% purity as measured by HPLC. A further 1.5 g of product with >94% purity was also isolated and could be purified further by recrystallization as above. The monomers may be polymerised by Stille or Suzuki polymerisation, e.g. Suzuki polymerisation as described in WO 00/53656.

The energy levels of the inventive materials were measured in solution by square-wave voltammetry as described above and the results are shown below in Table 1.

TABLE 1

| | | HOMO/ | LUMO/ |
|---|---|---|---|
| Material | Structure | eV | eV |
| Donor Example 1 | 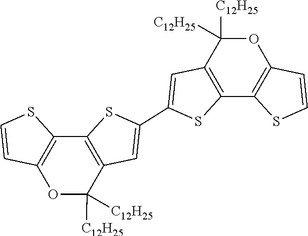 | −5.03 | −2.28 |
| Donor Example 2 | 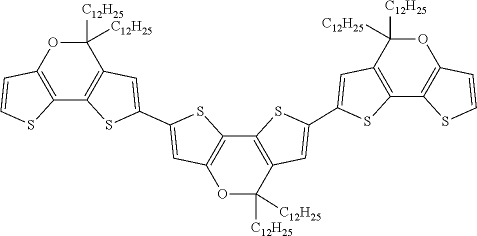 | −4.92 | −2.25 |
| Comparative Donor Example 1 | 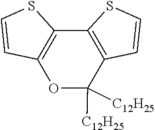 | −5.38 | −1.74 |

As shown in Table 1, a donor unit of formula (I) in which n=2 or 3 results in a smaller band gap, i.e. longer absorption wavelength, than a comparative compound in which n of the donor unit is 1, and the band gap reduces at with an increasing value of n.

### Modelling Examples

All modelling as described in these examples was performed using Gaussian09 software available from Gaussian using Gaussian09 with B3LYP (functional).

Compounds having a unit of Formula (I) and acceptor units in which bonds to adjacent repeat units are replaced with H were modelled and the results are set out in Table 2.

TABLE 2

| | | QC HOMO/ | QC LUMO/ |
|---|---|---|---|
| Unit | Structure | eV | eV |
| Donor Example 1 | 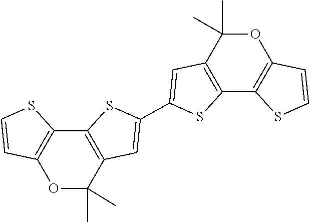 [SMILES: CC1(C)Oc2ccsc2-c2sc(-c3cc4c(s3)-c3sccc3OC4(C)C)cc21] | −4.62 | −1.76 |
| Donor Example 2 | 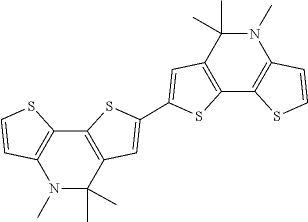 [SMILES: CN1c2ccsc2-c2sc(-c3cc4c(s3)-c3sccc3N(C)C4(C)C)cc2C1(C)C] | −4.19 | −1.50 |
| Acceptor Example 1 | 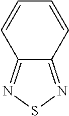 | −6.61 | −2.35 |
| Acceptor Example 2 | 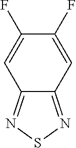 [SMILES: Fc1cc2nsnc2cc1F] | −6.95 | −2.60 |
| Acceptor Example 3 | 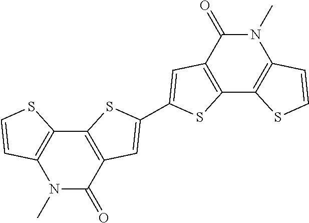 [SMILES: Cn1c(=O)c2cc(-c3cc4c(=O)n(C)c5ccsc5c4s3)sc2c2sccc21] | −5.10 | −2.01 |
| Comparative Donor | 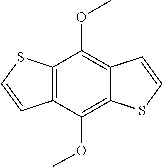 [SMILES: COc1c2ccsc2c(OC)c2ccsc12] | −5.29 | −1.05 |

As shown in Table 2, either of Donor Examples 1 and 2 may be used with any of Acceptor Examples 1-3 to form a D-A polymer.

HOMO and LUMO levels for model compounds having the following structure were modelled and the results are set out in Table 3:

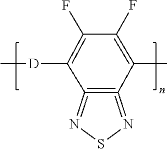

TABLE 3

| | HOMO/ | LUMO/ | Band gap/ |
|---|---|---|---|
| D | eV | eV | eV |
| 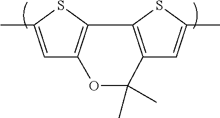 | −4.65 | −2.92 | 1.73 |
| 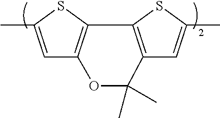 | −4.44 | −2.89 | 1.55 |
| 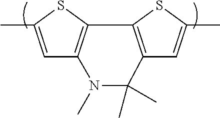 | −4.29 | −2.74 | 1.54 |
| 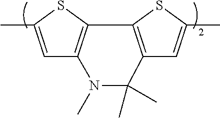 | −4.04 | −2.67 | 1.37 |

As shown in Table 3, a donor unit of formula (I) in which n=2 results in a smaller band gap, i.e. longer absorption wavelength, than comparative compounds in which n of the donor unit is 1.

1. A polymer comprising an electron-donating repeat unit of formula (I) and an electron-accepting repeat unit:
-(A)ₙ- (I)
wherein A in each occurrence is independently a group of formula (II): 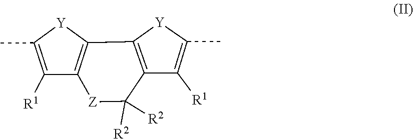
Y in each occurrence is independently O or S;
Z is O, S or NR³ wherein R³ is H or a substituent;
R¹ in each occurrence is independently H or a substituent;
R² in each occurrence is independently H or a substituent; and
n is at least 2.

2. The polymer according to claim 1 wherein n is 2.

3. The polymer according to claim 1 wherein each R¹ is H.

4. The polymer according to claim 1 wherein each R² is independently selected from the group consisting of:
linear, branched or cyclic C₁₋₂₀ alkyl wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1.

5. The polymer according to claim 1 wherein each Y is S.

6. The polymer according to a claim 1 wherein the electron-accepting repeat unit is selected from formulae (II)-(XIII): 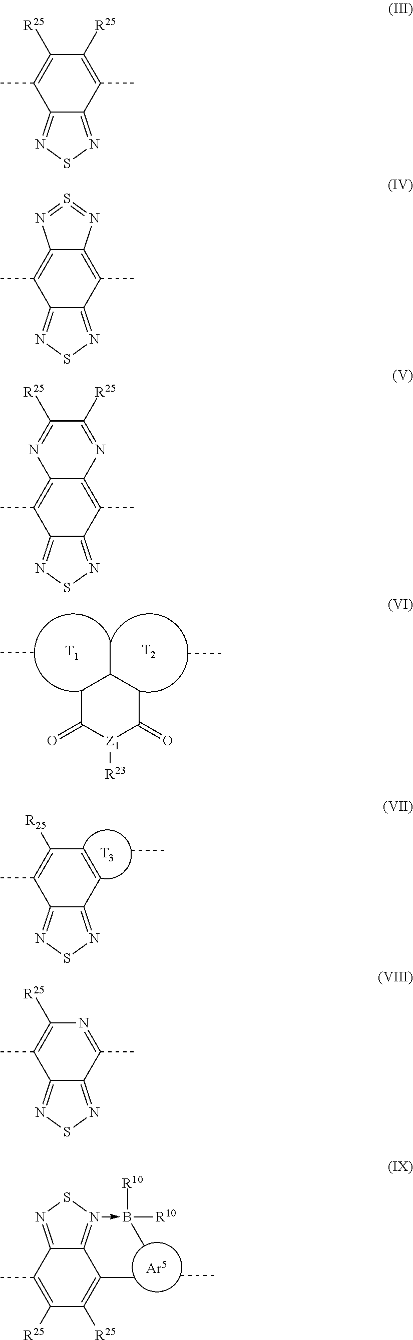 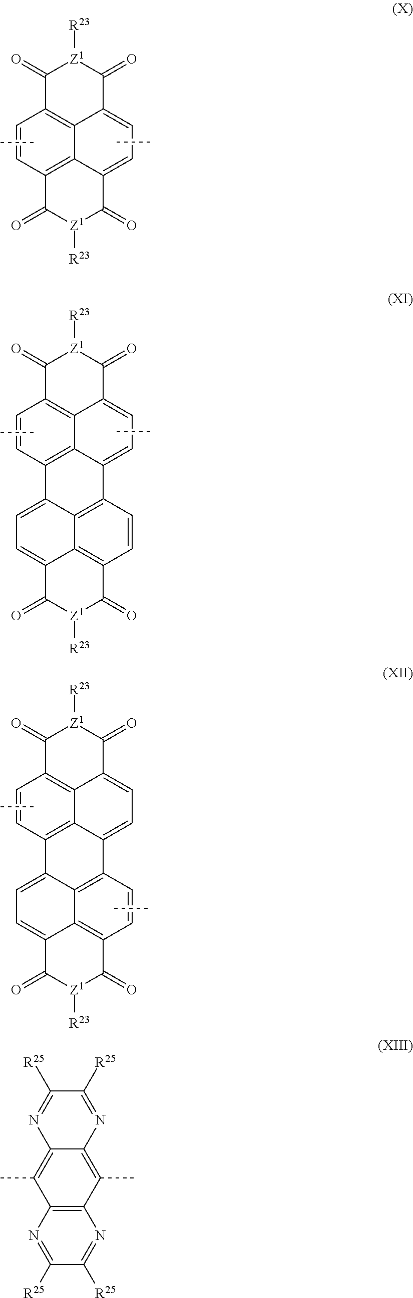
wherein R²³ in each occurrence is H or a substituent; R²⁵ in each occurrence is H or a substituent wherein two R²⁵ groups bound to adjacent carbon atoms may be linked to form a substituted or unsubstituted ring; Z¹ is N or P; T¹, T² and T³ each independently represent an aryl or a heteroaryl ring which may be fused to one or more further rings; R¹⁰ in each occurrence is a substituent; and Ar⁵ is an arylene or heteroarylene group which is unsubstituted or substituted with one or more substituents.

7. A composition comprising a polymer according to claim 1 and an electron-accepting material.

8. An organic electronic device comprising an active layer comprising compound according to claim 1.

9. An organic electronic device according to claim 8 wherein the organic electronic device is an organic photoresponsive device comprising a bulk heterojunction layer comprising the polymer comprising an electron-donating repeat unit of formula (I) and an electron-accepting material disposed between an anode and a cathode.

10. An organic electronic device according to claim 9 wherein the organic photoresponsive device is an organic photodetector.

11. A photosensor comprising a light source and an organic photodetector according to claim 10 wherein the photosensor is configured to detect light emitted from a light source.

12. A photosensor according to claim 11 wherein the light source emits light having a peak wavelength of at least 850 nm.

13. A formulation comprising a polymer according to claim 1 dissolved or dispersed in one or more solvents.

14. A method of forming an organic electronic device according to claim 8 wherein formation of the active layer comprises deposition of a polymer comprising an electron-donating repeat unit of formula (I) and an electron-accepting repeat unit dissolved or dispersed in one or more solvents onto a surface and evaporation of the one or more solvents.

15. A compound of formula (Im):
X-(A)ₙ-X (Im)
wherein:
X in each occurrence is independently selected from the group consisting of halogen, —OSO₂R⁴ wherein R⁴ is an optionally substituted C₁₋₁₂ alkyl group or optionally substituted aryl group; boronic acid and esters thereof; and —SnR⁵₃ wherein R⁵ independently in each occurrence is a C₁₋₁₂ hydrocarbyl group; and
A and n are as defined in claim 1.

16. A method of forming a polymer comprising an electron-donating repeat unit of formula (I) and an electron-accepting repeat unit comprising polymerisation of the compound of formula (Im) according to claim 15 and a compound for forming the electron-accepting repeat unit.
